(19) United States
(12) Patent Application Publication
(10) Pub. No.: US 2010/0278391 A1
(43) Pub. Date: Nov. 4, 2010

(54) Apparatus for behavior analysis and method thereof

(76) Inventors: Yung-Tai Hsu, Fongshan City (TW); JunWei Hsieh, Hsinchu City (TW); HongYuan Liao, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/546,400

(22) Filed: Oct. 12, 2006

(51) Int. Cl. G06T 7/00; A61B 5/00

(52) U.S. Cl. 382/106; 382/128; 600/300

(57) ABSTRACT

In the present invention, an apparatus for behavior analysis and method thereof is provided. In this apparatus, each behavior is analyzed and has its corresponding posture sequence through a triangulation-based method of triangulating the different triangle meshes. The two important posture features, the skeleton feature and the centroid context, are extracted and complementary to each other. The outstanding ability of posture classification can generate a set of key postures for coding a behavior sequence to a set of symbols. Then, based on the string representation, a novel string matching scheme is proposed to analyze different human behaviors even though they have different scaling changes. The proposed method of the present invention has been proved robust, accurate, and powerful especially in human behavior analysis.

### BACKGROUND OF THE INVENTION

1. Field of the Invention

The present invention relates to an apparatus for behavior analysis and the method thereof. More particularly, it relates especially to an apparatus, algorithm, and method thereof of behavior analysis, irregular activity detection and video surveillance for specific objects such as humankind.

2. Prior Arts

Behavior analysis, such as for humankind, is an important task in various applications like video surveillance, video retrieval, human interaction system, medical diagnosis, and so on. This result of behavior analysis can provide important safety information for users to recognize suspected people, to detect unusual surveillance states, to find illegal events, and thus to know all kinds of human daily activities from videos. In the past, there have been many approaches proposed for analyzing human behaviors directly from videos. For example, a visual surveillance system is proposed to model and recognize human behaviors using HMMs (Hidden Markov Models) and the trajectory feature. Also, a trajectory-based recognition system is proposed to detect pedestrians in outdoors and recognized their activities from multiple views based on a mixture of Gaussian classifier. In addition to trajectory, there are more approaches using human postures or body parts (such as head, hands, torso, and feet) to analyze human behaviors. For example, the complex 3-D models and multiple video cameras are used to extract 3-D voxels for 3-D posture analysis; the 3-D laser scanners and wavelet transform are used to recognize different 3-D human postures. Although 3D features are more useful for classifying human postures in more details, the inherent correspondence problem and the expensive cost of 3D acquisition equipments make them unfeasible for real-time applications. Therefore, more approaches are proposed for human behavior analysis based on 2D postures. For example, a probabilistic posture classification scheme is provided for classifying human behaviors, such as walking, running, squatting, or sitting. In addition, a 2D posture classification system is presented for recognizing human gestures and behaviors by HMM framework. Furthermore, a Pfinder system based on a 2D blob model is used for tracking and recognizing human behaviors. The challenge in incorporating 2D posture models in human behavior analysis is the ambiguities between the used model and real human behaviors caused by mutual occlusions between body parts, loose clothes, or similar colors between body articulations. Thus, in spite that the cardboard model is good for modeling articulated human motions, the requirement of body parts being well segmented makes it unfeasible for analyzing real human behaviors.

In order to solve this problem of body part segmentation, a dynamic Bayesian network for segmenting a body into different parts is based on the concept of blob to model body parts. This blob-based approach is very promising for analyzing human behaviors up to a semantic level, but it is very sensitive to illumination changes. In addition to blobs, another larger class of approaches to classify postures is based on the feature of human silhouette. For example, the negative minimum curvatures can be tracked along body contours to segment body parts and then recognized body postures using a modified ICP algorithm. Furthermore, a skeleton-based method is provided to recognize postures by extracting different skeleton features along the curvature changes of human silhouette. In addition, different morphological operations are exerted to extract skeleton features from postures and then recognized them using a HMM framework. The contour-based method is simple and efficient for making a coarse classification of human postures. However, it is easily disturbed by noise, imperfect contours, or occlusions. Another kind of approaches to classifying postures for human behavior analysis is using Gaussian probabilistic models. Such as in some methods, a probabilistic projection map is used to model each posture and performed a frame-by-frame posture classification to validate different human behaviors. This method used the concept of state-transition graph to integrate temporal information of postures for handling occlusions and making the system more robustly for handling indoors environments. However, the projection histogram used in this system is still not a good feature for posture classification owing to its dramatic changes under different lighting or viewing conditions.

### SUMMARY OF THE INVENTION

The present invention provides an apparatus and method thereof via a new posture classification system for analyzing different behaviors, such as for humankind, directly from video sequences using the technique of triangulation.

Via applying the present invention in the human behavior analysis, each human behavior consists of a sequence of human postures, which have different types and change rapidly at different time. For well analyzing the postures, first, the technique of Delaunay triangulation is used to decompose a body posture to different triangle meshes. Then, a depth-first search is taken to obtain a spanning tree from the result of triangulation. From the spanning tree, the skeleton features of a posture can be very easily extracted and further used for a coarse posture classification.

In addition to the skeleton feature, the spanning tree can also provide important information for decomposing a posture to different body parts like head, hands, or feet. Thus, a new posture descriptor, which is also called as a centroid context for describing a posture up to a semantic level, is provided to record different visual characteristics viewed from the centroids of the analyzed posture and its corresponding body parts. Since the two descriptors are complement to each other and can describe a posture not only from its syntactic meanings (using skeletons) but also its semantic ones (using body parts), the present invention can easily compare and classify all desired human postures very accurately. According to the outstanding discriminating abilities of these two descriptors of the present invention, a clustering technique is further proposed to automatically generate a set of key postures for converting a behavior to a set of symbols. The string representation integrates all possible posture changes and their corresponding temporal information. Based on this representation, a novel string matching scheme is then proposed for accurately recognizing different human behaviors. Even though each behavior has different time scaling changes, the proposed matching scheme still can recognize all desired behavior types very accurately. Extensive results reveal the feasibility and superiority of the present invention for human behavior analysis.

### BRIEF DESCRIPTION OF THE DRAWINGS

The various objects and advantages of the present invention will be more readily understood from the following detailed description when read in conjunction with the appended drawing, in which:

FIG. 1 is the flowchart of the proposed apparatus for analyzing different human behaviors.

FIG. 2(a) is the sampling of control points—Point with a high curvature.

FIG. 2(b) is the sampling of control points—Points with high curvatures but too close to each other.

FIG. 3 is the diagram of all the vertexes indexed anticlockwise such that the interior of V is located on their left.

FIG. 4 is the procedures of the divide-and-conquer algorithm.

FIG. 5 is the procedures of the skeleton extraction.

FIG. 6(a) is the triangulation result of a body posture—Input posture.

FIG. 6(b) is the triangulation result of a body posture—Triangulation result of FIG. 4(a).

FIG. 7(a) is the skeleton of human model—Original image.

FIG. 7(b) is the skeleton of human model—Spanning three of FIG. 5(a).

FIG. 7(c) is the skeleton of human model—Simple skeleton of FIG. 5(a).

FIG. 8 is the value of y is nonlinearly increased when x increases.

FIG. 9(a) is the distance transform of a posture—Triangulation result of a human posture.

FIG. 9(b) is the distance transform of a posture—Skeleton extraction of FIG. 7(a).

FIG. 9(c) is the distance transform of a posture—Distance map of FIG. 7(b).

FIG. 10 is the Polar Transform of a human posture.

FIG. 11(a) is the body component extraction—Triangulation result of a posture.

FIG. 11(b) is the body component extraction—A spanning tree of FIG. 9(a).

FIG. 11(c) is the body component extraction—Centroids of different body part extracted by taking off all the branch nodes.

FIG. 12(a) is the multiple centroid contexts using different numbers of sectors and shells—4 shells and 15 sectors.

FIG. 12(b) is the multiple centroid contexts using different numbers of sectors and shells—8 shells and 30 sectors.

FIG. 13 is the procedures of the skeleton extraction based on the FIG. 12(a) and FIG. 12(b).

FIG. 14(a) is the three kinds of different behaviors with different camera views—Walking.

FIG. 14(b) is the three kinds of different behaviors with different camera views—Picking up.

FIG. 14(c) is the three kinds of different behaviors with different camera views—Fall.

FIG. 15 is the result of key posture selection from four behavior sequences—walking, running, squatting, and gymnastics.

FIG. 16 is the recognition result of postures using multiple centroid contexts.

FIG. 17(a) is the irregular activity detection—Five key postures defining several regular human actions.

FIG. 17(b) is the irregular activity detection—A normal condition is detected.

FIG. 17(c) is the irregular activity detection—Triggering a warning message due to the detection of an irregular posture.

FIG. 18(a) is the irregular posture detection—Regular postures were detected.

FIG. 18(b) is the irregular posture detection—Irregular ones were detected due to the unexpected “shooting” posture.

FIG. 18(c) is the irregular posture detection—Regular postures were detected.

FIG. 18(d) is the irregular posture detection—Irregular ones were detected due to the unexpected “climbing wall” posture.

### DETAILED DESCRIPTION OF THE PREFERRED EMBODIMENTS

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

### OVERVIEW OF THE PRESENT INVENTION

In this invention, an apparatus for behavior analysis and method thereof, which is especially related to a novel triangulation-based system to analyze human behaviors directly from videos, is disclosed. The apparatus for behavior analysis of the present invention is based on a posture recognition technique. An apparatus for posture recognition comprises a triangulation unit and a recognition unit. The triangulation unit is responsible for dividing a posture captured by a background subtraction into several triangular meshes. Then, the recognition unit forms a spanning tree correspond to the triangular meshes from the triangulation unit. According to the postures analyzed via the apparatus for posture recognition, the apparatus for behavior analysis then receives the time-varied postures to build a behavior. The apparatus for behavior analysis comprises a clustering unit, coding unit and a matching unit. The clustering unit is able to merge the time-varied postures iteratively to obtain several key postures. Then, the coding unit translates the key postures into correspondent symbols, which are unscrambled through the matching unit as a behavior.

Furthermore, a system for irregular human action analysis based on the present invention introduced later comprises an action recognition apparatus and a judging apparatus, wherein the action recognition apparatus is in the basis of the abovementioned posture and behavior apparatus and is bale to integrate the behaviors clustered from the postures into an human action. According to the human action obtained, the judging apparatus identifies whether the human action is irregular or not. If the result of identification is regular, no alarm will be given. However, if the result of identification is irregular or suspicious, the warning unit is going to send an alarm to such as a surveillance system to arouse the guard or any correspondent person.

As shown in FIG. 1, the flowchart of the proposed apparatus is illustrated for analyzing different human behaviors. First of all, the method of background subtraction to extract different body postures from video sequences is used to obtain the posture boundaries. After that, a triangulation technique is then used for dividing a body posture to different triangle meshes. From the triangulation result, two important features including skeleton and centroid context (CC) are then extracted for posture recognition. The first feature, i.e., skeleton is used for a coarse search and the second feature, i.e., centroid context is for a finer classification to classify all postures with more syntactic meanings. In order to extract the skeleton feature, a graph search method is proposed to find a spanning tree from the result of triangulation. The spanning tree will correspond to a skeleton structure of the analyzed body posture. This method to extract skeleton features is more simple and effective and has more tolerances to noise than the contour tracing technique. In addition to skeleton, the tree can also provide important information for segmenting a posture to different body parts. According to the result of body part segmentation, the construction of a new posture descriptor, i.e., the centroid context is made for recognizing postures more accurately. This descriptor takes advantages of a polar labeling scheme to label each triangle mesh with a unique number. Then, for each body part, a feature vector, i.e., the centroid context can be constructed by recording all related features of each triangle mesh centroid according to this unique number. Then, the comparison of different postures would be more accurately by measuring the distance between their centroid contexts. After that, each posture will be assigned to a semantic symbol so that each human behavior can be converted and represented by a set of symbols. Based on this representation, a novel string matching scheme is then proposed for recognizing different human behaviors directly from videos. In the string-based method, the modification is required for the calculations of edit distance by using different weights to measure the operations of insertion, deletion, and replacement. Due to this modification, even though two behaviors have large scaling changes, the edit distance is still. The slow growth of edit distance can effectively tackle the time warping problem when aligning two strings. In what follows, firstly, the description of the technique of deformable triangulation is provided. The tasks of feature extraction, posture classification, and behavior analysis will be discussed thereinafter.

### Deformable Triangulations

The present invention assumes that all the analyzed video sequences are captured by a still camera. When the camera is static, the background of the analyzed video sequence can be constructed using a mixture of Gaussian functions. Then, different human postures can be detected and extracted by background subtraction. After subtraction, a series of simple morphological operations are then applied for noise removing. In this section, the description is stated for the technique of constrained Delaunay triangulation for dividing a posture to different triangle meshes. Then, two important posture features, i.e., the skeleton one and the centroid contexts can be extracted from the triangulation result for more accurate posture classification.

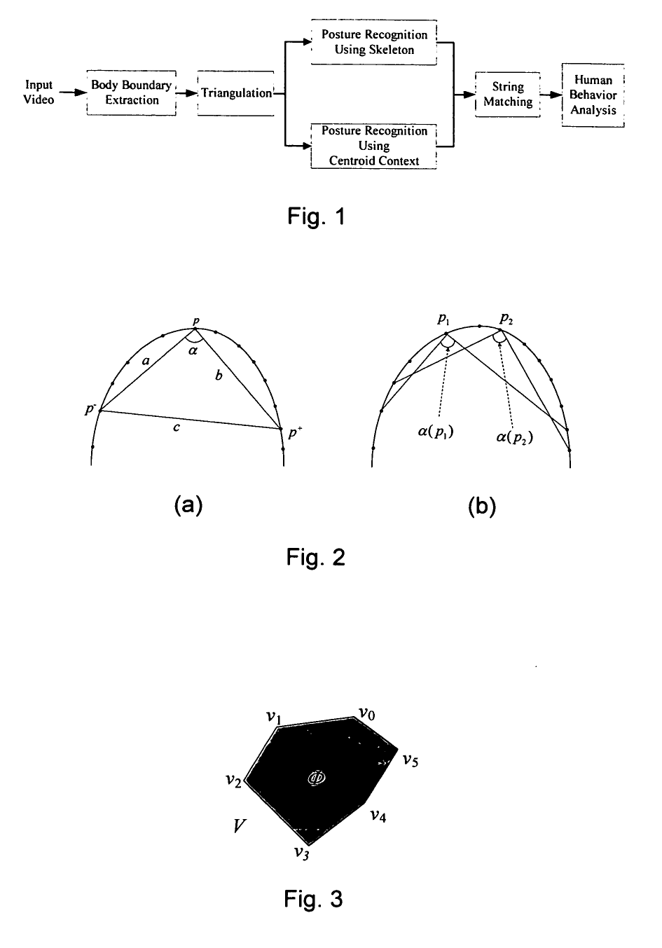

Assume that P is the analyzed posture which is a binary map extracted by image subtraction. To triangulate P, a set of control points should be extracted in advance along its contour. Let B be the set of boundary points extracted along the contour of P. In the present invention, a sampling technique is exerted to detect all the points with higher curvatures from B as the set of control points. Let α(p) be an angle of a point p in B. Shown in FIG. 2 (a), the sampling of control points with a high curvature is revealed, wherein the angle α can be determined by two specified points p⁺ and p⁻ which are selected from both sides of p along B and satisfy with the Eq. (1) below:

dₘᵢₙ≦|p−p⁺|≦dₘₐₓ and dₘᵢₙ≦|p−p⁻|≦dₘₐₓ, (1)

where dₘᵢₙ and dₘₐₓ are two thresholds and set to |B|/30 and |B|/20, respectively, and |B| the length of B. With p⁺ and p⁻, the angle α(p) can be decided as the Eq. (2) below:

$$α ( p ) = {cos}^{- 1} \frac{{‖ p - {p}^{+} ‖}^{2} + {‖ p - {p}^{-} ‖}^{2} - {‖ {p}^{-} - {p}^{+} ‖}^{2}}{2 ‖ p - {p}^{-} ‖ \times ‖ p - {p}^{+} ‖} . \qquad ( 2 )$$

If α is larger than a threshold T_{α}, i.e., 150°, p is selected as a control point. In addition to Eq. (2), it is expected that two control points should be far from each other. This enforces that the distance between any two control points should be larger than the threshold dₘᵢₙ defined in Eq. (1). Referring to FIG. 2(b), if two candidates p₁ and p₂ are close to each other, whose difference is not larger than the dₘᵢₙ, the one with a smaller angle α is chosen as the best control point.

Referring to the FIG. 3, the diagram of all the vertexes indexed anticlockwise is provided. In the present invention, assume that V is the set of control points extracted along the boundary of P. Each point in V is indexed anticlockwise and modulo by the size of V. If any two adjacent points in V are connected with an edge, V can be then considered as a planar straight line graph (PSLG), also referred as to a polygon. Based on this assumption, the present invention can use the technique of constrained Delaunay triangulation to divide V to different triangle meshes.

As what illustrated in FIG. 3, the assumption, that Φ is the set of interior points of V in R², is made. For a triangulation T⊂Φ, T is said to be a constrained Delaunay triangulation of V if under such a condition that each edge of V is an edge in T while each remaining edge e of T there exists a circle C such that the endpoints of e are on the boundary C. However, if a vertex in V is in the interior of C,V cannot be seen from at least one of the endpoints of e. More precisely, given three vertexes vᵢ, vⱼ, and vₖ in V, the triangle Δ(vᵢ,vⱼ,vₖ) belongs to the constrained Delaunay triangulation if and only if the following equations, Eq. (i) and Eq. (ii), are satisfied.

vₖεUᵢⱼ, where Uᵢⱼ={vεV|e(vᵢ,v)⊂Φ, e(vⱼ,v)⊂Φ} (i)

C(vᵢ,vⱼ,vₖ)∩Uᵢⱼ=Ø (ii)

where C is a circum-circle of vᵢ, vⱼ, and vₖ. That is, the interior of C(vᵢ,vⱼ,vₖ) includes no vertex vεUᵢⱼ.

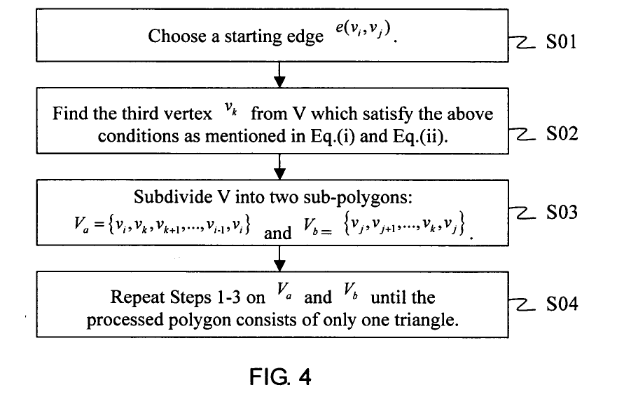

According to the abovementioned definition, a divide-and-conquer algorithm was developed to obtain the constrained Delaunay triangulation of V in O(n log n) time. The algorithm works recursively. When V contains only three vertexes, V is the result of triangulation. When V contains more than three vertexes, choose an edge from V and search the corresponding third vertex satisfying the properties disclosed in the Eq. (i) and Eq. (ii). Then subdivide V to two sub-polygons Vₐ and V_{b}. The same division procedure is recursively applied to Vₐ and V_{b} until only one triangle is included in the processed polygon. Details of the algorithm perform the following four steps and are shown in FIG. 4:
S01: Choose a starting edge e(vᵢ,vⱼ).
S02: Find the third vertex vₖ from V which satisfy the above conditions as mentioned in Eq. (i) and Eq. (ii).
S03: Subdivide V into two sub-polygons: Vₐ={vᵢ, vₖ, vₖ₊₁, . . . , vᵢ₋₁, vᵢ} and V_{b}={vⱼ, vⱼ₊₁, . . . , vₖ, Vⱼ}.

S04: Repeat Steps 1-3 on Vₐ and V_{b} until the processed polygon consists of only one triangle.

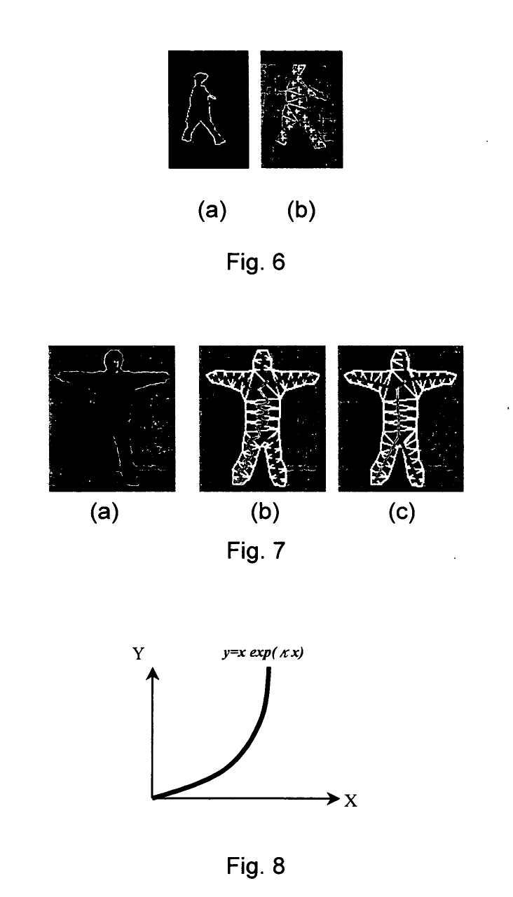

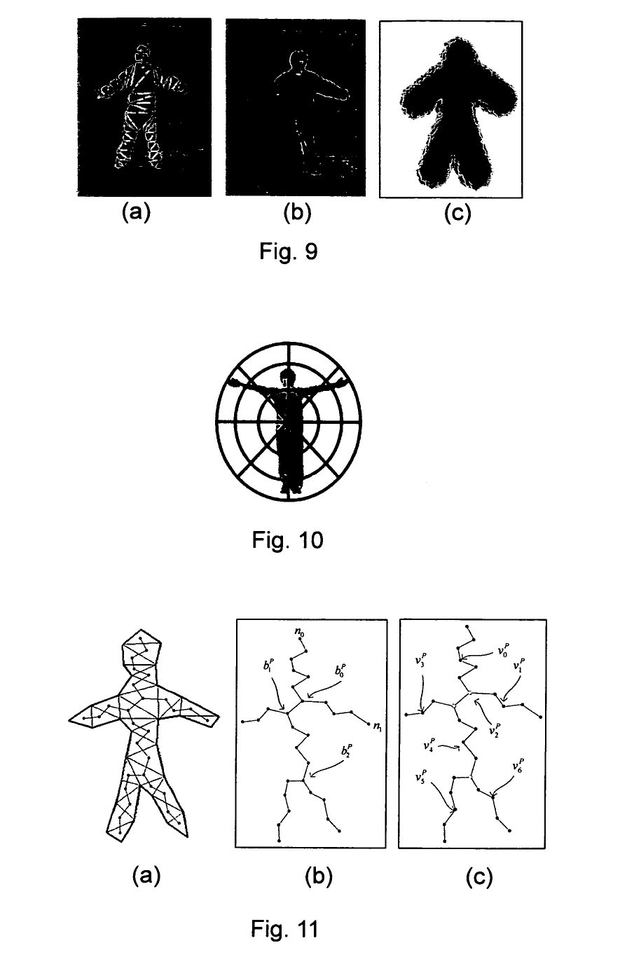

At last, the FIG. 6(a) and FIG. 6(b) show one example of triangulation analysis of a human posture with the input posture and the final result, respectively.

### Skeleton-based Posture Recognition

In the present invention, two important posture features are extracted from the result of triangulation, i.e., the skeleton and centroid context ones. This section will discuss the method of skeleton extraction using the triangulation technique. Traditional methods to extract skeleton features, which different feature points with negative minimum curvatures are extracted along the body contours of a posture for constructing its body skeletons, are mainly based on body contours In order to avoid the drawbacks of the heuristic and noise-disturbed skeleton construction, a graph search scheme is disclosed to find a spanning tree which corresponds to a specified body skeleton. Thus, in the present, different postures can be recognized using their skeleton features.

### Triangulation-based Skeleton Extraction

In the section of deformable triangulations, a technique is presented to triangulate a human body to different triangle meshes. By connecting all the centroids of any two connected meshes, a graph will be formed. Though the technique of depth first search, the desired skeleton from this graph for posture recognition is found.

Assume that P is a binary posture. According to the technique of triangulation, P will be decomposed to a set Ω_{P} of triangle meshes, i.e.,

$${Ω}_{P} = {\{ {T}_{i} \}}_{i = 0 , 1 , … , {N}_{{T}_{P}} - 1} .$$

Each triangle mesh Tᵢ in Ω_{P} has the centroid C_{T}_{ᵢ}. One common edge is shared if two given triangle meshes Tᵢ and Tⱼ are connected. According to this connectivity, P can be converted to an undirected graph G_{P}, where all centroids C_{T}_{ⱼ} in Ω_{P} are the nodes in G_{P} and an edge exists between C_{T}_{ᵢ} and C_{T}_{ⱼ} if Tᵢ and Tⱼ are connected. The degree of a node mentioned here is defined the number of edges in it. Thus, based on the above definitions, a graph searching scheme on G_{P} is revealed for extracting its skeleton feature. First, a node H, whose degree is one and position is the highest for all the nodes in G_{P}, is selected, where H is defined the head of P. Then, starting from H, a depth first spanning tree is found. In this tree, all the leaf nodes Lᵢ correspond to different limbs of P. The branching nodes Bᵢ (whose degrees are three in G_{P}) are the key points used for decomposing P to different body parts like hands, foots, or torso. Let C_{P} be the centroid of P and U the union of H, C_{P}, Lᵢ and Bᵢ. The skeleton S_{P} of P can be extracted by connecting any two nodes in U if they are connected, i.e., a path existing between them, and without passing other nodes in U. The path can be easily found and checked from the spanning tree of P.
Further, in what follows, details of the algorithm for skeleton extraction are summarized.

### Triangulation-Based Simple Skeleton Extraction Algorithm (TSSE):

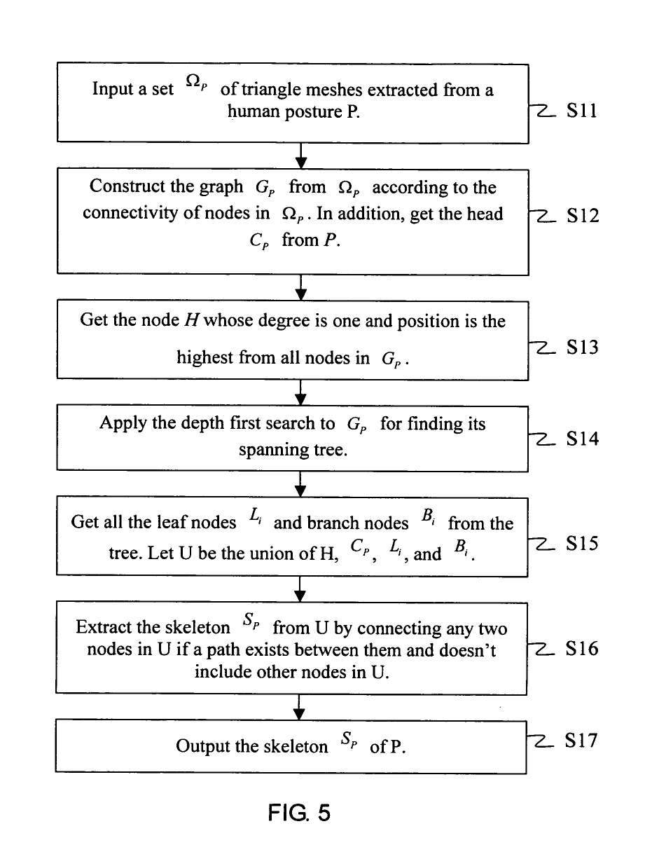

First, the procedures of the triangulation-based simple skeleton extraction shown in FIG. 5 are listed below:
S11: Input a set Ω_{P} of triangle meshes extracted from a human posture P.
S12: Construct the graph G_{P} from Ω_{P} according to the connectivity of nodes in Ω_{P}. In addition, get the centroid C_{P} from P.
S13: Get the node H whose degree is one and position is the highest from all nodes in G_{P}.
S14: Apply the depth first search to G_{P} for finding its spanning tree.
S15: Get all the leaf nodes Lᵢ and branch nodes Bᵢ from the tree. Let U be the union of H, C_{P}, Lᵢ, and Bᵢ.
S16: Extract the skeleton S_{P} from U by connecting any two nodes in U if a path exists between them and doesn't include other nodes in U.
S17: Output the skeleton S_{P} of P.

Actually, the spanning tree of P obtained by the depth search also is a skeleton feature. Referring to FIG. 8(a)-(c), the skeleton of human model in the original posture, its spanning tree and the corresponding TSSE algorithm are illustrated respectively. It is clear to find that FIG. 8(b) is also a skeleton of FIG. 8(a). In the present invention, the skeleton obtained by connecting all branch nodes is called as “simple skeleton” due to its simple shape. The spanning tree is served as the “complex skeleton” of a posture due to its irregular shape. The complex skeleton performs better than the simple one.

### Posture Recognition Using Skeleton

In the previous section, a triangulation-based method has been proposed for extracting skeleton features from a body posture. Assume that S_{P} and S_{D} are two skeletons extracted from a testing posture P and another posture D in database, respectively. In what follows, a distance transform is applied to converting each skeleton to a gray level image. Based on the distance maps, the similarity between S_{P} and S_{D} can be compared.

First, assume that DT_{S}_{_{P}} is the distance map of S_{P}. The value of a pixel r in DT_{S}_{_{P}} is its shortest distance to all foreground pixels in S_{P} and satisfied with Eq. (3) below:

$${DT}_{{S}_{P}} ( r ) = {min}_{q ∈ {S}_{P}} d ( r , q ) , \qquad ( 3 )$$

where d(r,q) is the Euclidian distance between r and q. In order to enhance the strength of distance changes, Eq. (3) is further modified as the Eq. (4):

$${DT}_{{S}_{P}} ( r ) = {min}_{q ∈ {S}_{p}} d ( r , q ) \times exp ( κ d ( r , q ) ) , \qquad ( 4 )$$

where κ=0.1. As shown in FIG. 8, when x increases more, the value of y will increase more rapidly than x. The distance of distance maps between P and D is defined by the Eq. (5):

$${d}_{skeleton} ( {S}_{P} , {S}_{D} ) = \frac{1}{\vert {DT}_{{S}_{P}} \vert} {\sum }_{r} \vert {DT}_{{S}_{P}} ( r ) - {DT}_{{S}_{D}} ( r ) \vert , \qquad ( 5 )$$

where |DT_{S}_{_{P}}| is the image size of DT_{S}_{_{P}}. When calculating Eq. (5), S_{P} and S_{D} are normalized to a unit size and their centers are set to the originals of DT_{S}_{_{P}} and DT_{S}_{_{D}}, respectively. Respectively, FIG. 10(a)-(c) shows one result of distance transform of a posture after skeleton extraction in the original posture, the result of skeleton extraction, and the distance map of FIG. 10(b).

### Posture Recognition Using Centroid Context

In the previous section, a skeleton-based method is proposed to analyze different human postures from video sequences. This method has advantages in terms of simplicity of use and efficiency in recognizing body postures. However, skeleton is a coarse feature to represent human postures and used here for a coarse search in posture recognition. For recognizing different postures more accurately, this section will propose a new representation, i.e., the centroid context for describing human postures in more details.

### Centroid Context of Postures

The present invention provides a shape descriptor to finely capture postures' interior visual characteristics using a set of triangle mesh centroids. Since the triangulation result may vary from one instance to another, the distribution is identified over relative positions of mesh centroids as a robust and compact descriptor. Assume that all the analyzed postures are normalized to a unit size. Similar to the technique used in shape context, a uniform sample in log-polar space is used for labeling each mesh, where m shells are used for quantifying radius and n sectors for quantifying angle. Then, the total number of bins used for constructing the centroid context is m×n. For the centroid r of a triangle mesh in an analyzed posture, a vector histogram is constructed and satisfied with Eq. (6) below:

hᵣ=(hᵣ(1), . . . , hᵣ(k), . . . , hᵣ(mn)). (6)

In this embodiment, hᵣ(k) is the number of triangle mesh centroids resides in the kth bin by considering r as the reference original. The relationship of hᵣ(k) and r is shown as Eq. (7):

hᵣ(k)=#{q≠r,(q−r)εbin^{k}}, (7)

where bin^{k} is the kth bin of the log-polar coordinate. Then, the distance between two histograms hᵣ_{ᵢ}(k) and hᵣ_{ⱼ}(k) can be measured by the normalized intersection shown in Eq. (8):

$$C ( {r}_{i} , {r}_{j} ) = 1 - \frac{1}{{N}_{mesh}} {\sum }_{k = 1}^{{K}_{bin}} min \{ {h}_{{r}_{i}} ( k ) , {h}_{{r}_{j}} ( k ) \} , \qquad ( 8 )$$

where K_{bin} is the number of bins and Nₘₑₛₕ the number of meshes fixed in all the analyzed postures. With the help of Eq. (6) and Eq. (7), a centroid context can be defined to describe the characteristics of a posture P.

In the previous section, a tree searching method is presented to find a spanning tree T_{dfs}^{P} from a posture P according to its triangulation result. Referring to FIG. 11(a)-(c), the triangulation result of a posture body component extraction is illustrated in FIG. 11(a); the spanning tree corresponding to FIG. 11(a) is shown in FIG. 11(b); the centroids of different body part are shown in FIG. 11(c). The tree T_{dfs}^{P} captures the skeleton feature of P. In the present invention, a node is called as a branch node if it has more than one child. According to this definition, there are three branch nodes in FIG. 11(b), i.e., b₀^{P}, b₁^{P}, and b₂^{P}. If all the branch nodes are removed from T_{dfs}^{P}, T_{dfs}^{P} will be decomposed into different branch paths pathᵢ^{P}. Then, by carefully collecting the set of triangle meshes along each path, it is clear that each path pathᵢ^{P} will correspond to one of body parts in P. For example, in FIG. 11(b), if b₀^{P} is removed from T_{dfs}^{P}, two branch paths are formed, that is, the one from node n₀ to b₀^{P} and the other one from b₀^{P} to node n₁. The first one will correspond to the head and neck of P and the second one correspond to the hand of P. In some examples like the path from b₀^{P} to b₁^{P}, it does not exactly correspond to a high-level semantic body component. However, if the path length is further considered and constrained, the issue of over-segmentation can be easily avoided.

Given a path pathᵢ^{P}, a set Vᵢ^{P} of triangle meshes can be collected along pathᵢ^{P}. Let cᵢ^{P} be the centroid of the triangle mesh, which is the closest to the center of this set of triangle meshes. As shown in FIG. 11(c), cᵢ^{P} is the centroid extracted from the path beginning from n₀ to b₀^{P}. The corresponding histogram h_{c}_{ᵢ}_{^{P}}(k) of the given centroid cᵢ^{P} can be obtained via using Eq. (7). Assume that the set of these path centroids is V^{P}, further, based on V^{P}, the centroid context of P is defined as Eq. (9) below:

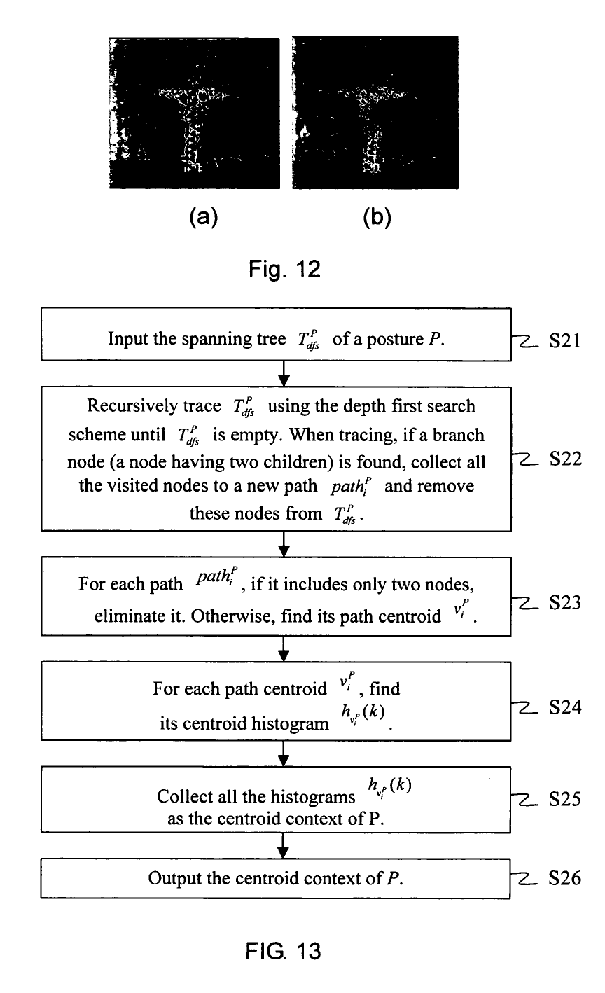

P={h_{c}_{ᵢ}_{^{P}}}_{i=0, . . . , |V}_{^{P}}_{|-1}, (9)

where |V^{P}| is the number of elements in V^{P}. According to FIG. 12(a) and FIG. 12(b), two embodiments of multiple centroid contexts when the number of shells and sectors is set to (4, 15) and (8, 30), are provided respectively. In addition, the centroid contexts are extracted from the head and the posture center, respectively. Given two postures P and Q, the distance between their centroid contexts is measured by the Eq. (10):

$${d}_{cc} ( P , Q ) = \frac{1}{2 \vert {V}^{P} \vert} {\sum }_{i = 0}^{\vert {V}^{P} \vert - 1} {w}_{i}^{P} {min}_{0 \leq j < \vert {V}^{P} \vert} C ( {c}_{i}^{P} , {c}_{j}^{Q} ) + \frac{1}{2 \vert {V}^{Q} \vert} {\sum }_{j = 0}^{\vert {V}^{Q} \vert - 1} {w}_{j}^{Q} {min}_{0 \leq i < \vert {V}^{Q} \vert} C ( {c}_{i}^{P} , {c}_{j}^{Q} ) \qquad ( 10 )$$

where wᵢ^{P} and wⱼ^{Q} are area ratios of the ith and jth body parts reside in P and Q, respectively. Based on Eq. (10), an arbitrary pair of postures can be compared. In what follows, the algorithm shown in FIG. 13 for finding the centroid context of a posture P is summarized.
S21: Input the spanning tree T_{dfs}^{P} of a posture P.
S22: Recursively trace T_{dfs}^{P} using the depth first search scheme until T_{dfs}^{P} is empty. When tracing, if a branch node (a node having two children) is found, collect all the visited nodes to a new path pathᵢ^{P} and remove these nodes from T_{dfs}^{P}.
S23: For each path pathᵢ^{P} if it includes only two nodes, eliminate it. Otherwise, find its path centroid vᵢ^{P}.
S24: For each path centroid vᵢ^{P}, find its centroid histogram hᵥ_{ᵢ}_{^{P}}(k) using Eq. (7).
S25: Collect all the histograms hᵥ_{ᵢ}_{^{P}}(k) as the centroid context of P.
S26: Output the centroid context of P.

### Posture Recognition Using Skeleton and Centroid Context

The skeleton feature and centroid context of a given posture can be extracted via using the techniques described in sections of triangulation-based skeleton extraction and centroid context of postures, respectively. Then, the distance between any two postures can be measured using Eq. (5) (for skeleton) or Eq. (10) (for centroid context). The skeleton feature is for a coarse search and the centroid context feature is for a fine search. For receiving better recognition results, the two distance measures should be integrated together. We use a weighted sum to represent the total distance, it is represented as follows:

Error(P,Q)=wdₛₖₑₗₑₜₒₙ(P,Q)+(1−w)d_{CC}(P,Q), (11)

where Error(P,Q) is the total distance between two postures P and Q and w is a weight used for balancing dₛₖₑₗₑₜₒₙ(P,Q) and d_{CC}(P,Q). is the integrated distance between two postures P and Q and w a weight for balancing the two distances dₛₖₑₗₑₜₒₙ(P,Q) and d_{cc}(P,Q). However, this weight w is difficult to be automatically decided, and even, different settings of w will lead to different performances and accuracies of posture recognition.

### Behavior Analysis Using String Matching

In the present invention, each behavior is represented by a sequence of postures which will change at different time. For well analyzing, the sequence is converted into a set of posture symbols. Then, different behaviors can be recognized and analyzed through a novel string matching scheme. This analysis requires a process of key posture selection. Therefore, in what follows, a method is disclosed to automatically select a set of key postures from training video sequences. Then, a novel scheme string matching is proposed for effective behavior recognition.

### Key Posture Selection

In the present invention, different behaviors are directly analyzed from videos. For a video clip, there should be many redundant and repeated postures, which are not properly used for behavior modeling. Therefore, a clustering technique is used to select a set of key postures from a collection of training video clips.

Assuming that all the postures have been extracted from a video clip, each frame has only one posture and P, is the posture extracted from the tth frame. Two adjacent postures Pₜ₋₁ and Pₜ with a distance dₜ calculated via using Eq. (10), where w is set to 0.5, are provided in this embodiment. Based on the assumption that T_{d} is the average vale of dₜ for all pairs of adjacent postures, a posture change event occurs for a posture Pₜ when dₜ is greater than 2T_{d}. Through collecting all the postures, which hit an event of posture change, a set S_{KPC} of key posture candidates can be got. However, S_{KPC} still contains many redundant and repeated postures, which will degrade the effectiveness of behavior modeling. To tackle this problem, a clustering technique will be proposed for finding another better set of key postures.

Initially, each element eᵢ in S_{KPC} forms a cluster zᵢ. Then, two cluster elements zᵢ and zⱼ in S_{KPC} are selected and the distance between these two cluster elements is defined by Eq. (12):

$${d}_{cluster} ( {z}_{i} , {z}_{j} ) = \frac{1}{\vert {z}_{i} \vert \vert {z}_{j} \vert} {\sum }_{{e}_{m} ∈ {z}_{i}} {\sum }_{{e}_{n} ∈ {z}_{j}} Error ( {e}_{m} , {e}_{n} ) , \qquad ( 12 )$$

where Error(.,.) is defined in Eq. (11) and |zₖ| the number of elements in zₖ. According to Eq. (12), an iterative merging scheme is performed to find a compact set of key postures from S_{KPC}. zᵢ^{t} and Z^{t} are the ith cluster and the collection of all these clusters zᵢ^{t} at the tth iteration. At each iteration, a pair of clusters zᵢ^{t} and zⱼ^{t} are chosen and the distance, d_{cluster}(zᵢ,zⱼ), between zᵢ^{t} and zⱼ^{t} is the minimum for all pairs in Z^{t}, which is satisfied with the following Eq. (13):

$$( {z}_{i} , {z}_{j} ) = arg {min}_{( \overline{-} m \cdot \overline{-} n )} {d}_{cluster} ( {z}_{m} , {z}_{n} ) , for all {z}_{m} ∈ {Z}^{′} , {z}_{n} ∈ {Z}^{′} , and {z}_{m} \neq {z}_{n} . \qquad ( 13 )$$

As the abovementioned, when d_{cluster}(zᵢ,zⱼ) is less than T_{d}, the two clusters zᵢ^{t} and zⱼ^{t} are merged together for forming a new cluster and thus constructing a new collection Z^{t+1} of clusters. The merging process is iteratively performed until no pair of clusters is merged. Based on the assumption that Z̅ is the final set of clusters after merging, the formation of the ith z̅ⁱ cluster in Z̅ can be used to extract a key posture eᵢ^{key}, which satisfies the Eq. (14):

$${e}_{i}^{key} = arg {min}_{{e}_{m} ∈ {\overline{z}}_{i}} {\sum }_{{e}_{n} ∈ {\overline{z}}_{i}} Error ( {e}_{m} , {e}_{n} ) . \qquad ( 14 )$$

As referring to Eq. (14) and checking all clusters in Z̅, the set S_{KP}, of key postures, i.e., S_{KP}={eᵢ^{key}} can be constructed for further action sequence analysis.

### Behavior Recognition Using String Matching

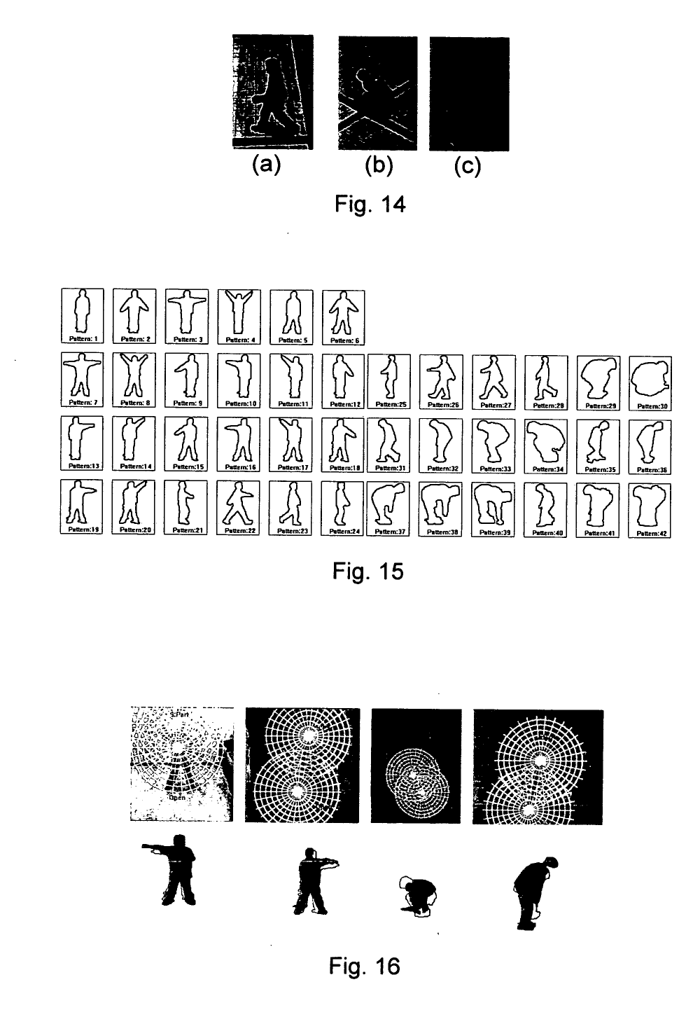

According to the result of key posture selection and posture classification, different behaviors with strings can be modeled. For example, in FIG. 14, there are three kinds of behaviors including walking, picking up, and fall. The symbols ‘s’ and ‘e’ denote the starting and ending points of a behavior respectively. Then, the behavior in FIG. 14(a) can be presented by “swwwe”, the one in FIG. 14(b) represented by “swwppwwe”, and the one in FIG. 14(c) represented by “swwwwffe”, where ‘w’ is for a walking posture, p′ for a picking-up posture and ‘f’ for a fall one. According to this converting, different behaviors can be well represented and compared using a string matching scheme.

Assume that Q and D are two behaviors whose string representations are S_{Q} and S_{D}, respectively. The edit distance between S_{Q} and S_{D}, which is defined as the minimum number of edit operations required to change S_{Q} into S_{D}, is used to measure the dissimilarity between Q and D. The operations include replacements, insertions, and deletions. For any two strings S_{Q} and S_{D}, the definition of D_{S}_{_{Q}}_{,S}_{_{D}}^{e}(i,j) is referred as the edit distance between S_{Q}[0 . . . i] and S_{D}[0 . . . j]. That is, D_{S}_{_{Q}}_{,S}_{_{D}}^{e}(i,j) is the minimum edit operations needed to transform the first (i+1) characters of S_{Q} into the first (j+1) characters of S_{D}. In addition, α(i,j) is a function, which is 0 if S_{Q}(i)=S_{D}(j) and 1 if S_{Q} (i)≠S_{D}(j), and then, D_{S}_{_{Q}}_{,S}_{_{D}}^{e}(0,0)=α(0,0), D_{S}_{_{Q}}_{,S}_{_{D}}^{e}(i,0)=i+α(0,0), and D_{S}_{_{Q}}_{,S}_{_{D}}^{e}(0,j)=j+α(0,0) can be got. Furthermore, the value of D_{S}_{_{Q}}_{,S}_{_{D}}^{e}(i,j) can be easily calculated with the recursive form as shown in Eq. (15):

D_{S}_{_{Q}}_{,S}_{_{D}}^{e}(i,j)=min[D_{S}_{_{Q}}_{,S}_{_{D}}^{e}(i−1,j)+1, D_{S}_{_{Q}}_{,S}_{_{D}}^{e}(i,j−1)+1, D_{S}_{_{Q}}_{,S}_{_{D}}^{e}(i−1,j−1)+α(i,j)], (15)

where the “insertion”, “deletion”, and “replacement” operations are the transition from cell (i−1,j) to cell (i,j), the one from cell (i,j−1) to cell (i,j), and the other one from cell (i−1,j−1) to cell (i,j), respectively.
Assume that the query Q is a walking video clip whose string representation is “swwwwwwe”. However, the string representation of Q is different to the one of FIG. 14(a) due to the time-scaling problem of videos. According to Eq. (14), the edit distance between Q and FIG. 14(a) is 3 while the one between Q and FIG. 14(b) and the one between Q and FIG. 14(c) are both equal to 2. However, Q is more similar to FIG. 14(a) than FIG. 14 (b) and FIG. 14 (c). Clearly, Eq. (14) cannot directly be applied in behavior analysis and should be modified. As described before, two similar strings often have scaling changes. This scaling change will lead to a large value of edit distance between them if the costs to perform each edit operation are equal. Thus, a new edit distance should be defined for tackling this problem. So, if C_{i,j}^{I}, C_{i,j}^{R} and C_{i,j}^{D} are the costs of the “insertion”, “replacement”, and “deletion” operations performed in the ith and jth characters of S_{Q} and S_{D}, respectively, then, Eq. (14) can be rewritten as Eq. (16) below:

D_{S}_{_{Q}}_{,S}_{_{D}}^{e}(i,j)=min[D_{S}_{_{Q}}_{,S}_{_{D}}^{e}(i−1,j)+C_{i,j}^{I}, D_{S}_{_{Q}}_{,S}_{_{D}}^{e}(i,j−1)+C_{i,j}^{D}, D_{S}_{_{Q}}_{,S}_{_{D}}^{e}(i−1,j−1)+C_{i,j}^{R}]. (16)

In the present invention, the “replacement” operation is considered more important than the “insertion” and “deletion” ones since a replacement means a change of posture type. Thus, the costs of “insertion” and “deletion” are chosen cheaper than the one of “replacement” and assumed to be ρ, where ρ<1. According to this, when an “insertion” is adopted in calculating the distance D_{S}_{_{Q}}_{,S}_{_{D}}^{e}(i,j), its cost will be ρ if S_{Q}[i]=S_{D}[j]; otherwise the cost will be 1. This implies that C_{i,j}^{I}=ρ+(1ρ)α(i,j). Similarly, for the “deletion” operation, C_{i,j}^{D}=ρ+(1−ρ)α(i,j) is obtained. However, if S_{Q}[i]≠S_{D}[j], it is impossible to choose “replacement” as the next operation for the costs of “insertion” and “deletion” are smaller than “replacement”. This problem can be easily solved by setting the cost C_{i,j}^{R} as α(i−1,j−1), that is, the characters S_{Q}[i] and S_{Q}[j] are not compared when calculating D_{S}_{_{Q}}_{,S}_{_{D}}^{e}(i,j) but will be done when calculating D_{S}_{_{Q}}_{,S}_{_{D}}^{e}(i+1,j+1). Since the same ending symbol ‘e’ appears in both strings S_{Q} and S_{D}, the final distance D_{S}_{_{Q}}_{,S}_{_{D}}^{e}(|S_{Q}|−1, |S_{D}|−1) is equal to its previous value D_{S}_{_{Q}}_{,S}_{_{D}}^{e}(|S_{Q}|−2, |S_{D}|−2). Thus, the delay of comparison will not cause any errors but will increase the costs of “insertion” and “deletion” if wrong edit operations are chosen. Then, the precise form of Eq. (15) is modified as Eq. (17) below:

D_{S}_{_{Q}}_{,S}_{_{D}}^{e}(i,j)=min[D_{S}_{_{Q}}_{,S}_{_{D}}^{e}(i−1,j)+C_{i,j}^{I}, D_{S}_{_{Q}}_{,S}_{_{D}}^{e}(i,j−1)+C_{i,j}^{D}, D_{S}_{_{Q}}_{,S}_{_{D}}^{e}(i−1,j−1)+α(i−1,j−1)], (17)

where C_{i,j}^{I}=ρ+(1−ρ)α(i−1,j) and C_{i,j}^{D}=ρ+(1−ρ)α(i,j−1). In the present invention, one “replacement” operation means a change of posture type. It implies that ρ should be much smaller than 1 and thus set to 0.1 in this invention. The setting makes the method proposed be nearly scaling-invariant.

### Performance of the Invention

In order to analyze the performance of our approach, a test database containing thirty thousands of postures, which come from three hundreds of video sequences, was constructed. Each sequence records a specific behavior. FIG. 15 shows the results of key posture selection extracting from the sequences of walking, running, squatting, and gymnastics while FIG. 16 shows the recognition result when the descriptor of multiple CC was used.

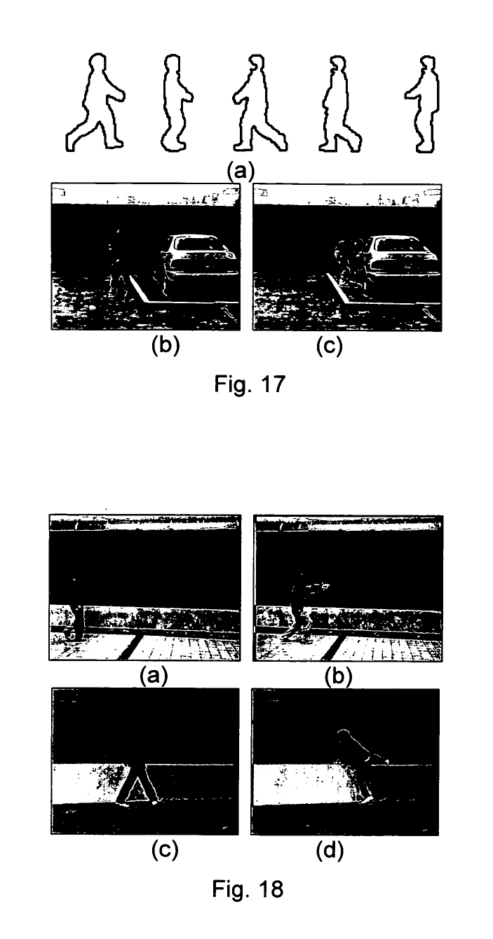

In addition to posture classification, the proposed method can be also used to analyze irregular or suspicious human actions for safety guarding. The task first extracts a set of “normal” key postures from training video sequences for learning different human “regular” actions like walking or running. Then, different input postures can be judged whether they are “regular”. If the irregular or suspicious postures appear continuously, an alarm message will be trigged for safety warming. For example, in FIG. 17(a), a set of normal key postures is extracted from a walking sequence. Then, based on FIG. 17(a) and the classification of the posture in FIG. 17(b) is revealed for a regular condition. However, the posture in FIG. 17(c) was classified “irregular” since the person had a suspicious posture (opening the car door or a stealing attempt). Then, a red area will be drawn for alarming a warning message. Further, FIG. 18 shows another two embodiments of irregular posture detection. The postures in FIG. 18(a) and FIG. 18(c) are recognized “normal” since they are similar to the postures in FIG. 17(a). However, the ones in FIG. 18(b) and FIG. 18(d) are classified “irregular” since the persons had “shooting” and “climbing wall” postures. The function of irregular posture detection can provide two advantages for building a video surveillance system: one is the saving of storage memories and the other is the significant reduction of browsing time since only the frames with red alarms should be saved and browsed.

In the final embodiment, the performance of the proposed algorithm for behavior analysis with string matching is disclosed. The present invention collects three hundreds of behavior sequences for measuring the accuracy and robustness of behavior recognition using our proposed string matching method. Ten kinds of behavior types are included in this set of behavior sequences. Thus, each behavior type collects thirty testing video sequences for behavior analysis. Table 1 lists the details of comparisons among different behavior categories. Each behavior sequence has different scaling changes and wrong posture types caused by recognition errors. However, the proposed string method of the present invention still performed well to recognize all behavior types.

TABLE 1

| | Behavior Types | | | | | | | | | |
|---|---|---|---|---|---|---|---|---|---|---|
| Query | Gy | Wa | Sq | St | Si | La | Fa | Pi | Ju | Cl |
| Gymnastics | 44 | 1 | 0 | 0 | 0 | 0 | 0 | 0 | 0 | 0 |
| Walk | 0 | 43 | 0 | 0 | 0 | 0 | 0 | 0 | 2 | 0 |
| Squat | 0 | 0 | 40 | 0 | 0 | 0 | 0 | 5 | 0 | 0 |
| Stoop | 0 | 0 | 0 | 41 | 0 | 0 | 0 | 4 | 0 | 0 |
| Sitting | 0 | 0 | 0 | 0 | 45 | 0 | 0 | 0 | 0 | 0 |
| Laying | 0 | 0 | 0 | 0 | 0 | 43 | 1 | 0 | 0 | 1 |
| Fallen | 0 | 0 | 0 | 0 | 0 | 1 | 42 | 0 | 0 | 2 |
| Picking up | 0 | 0 | 2 | 1 | 0 | 0 | 0 | 42 | 0 | 0 |
| Jumping | 0 | 1 | 0 | 0 | 0 | 0 | 0 | 0 | 44 | 0 |
| Climbing | 0 | 0 | 0 | 0 | 0 | 1 | 1 | 0 | 0 | 43 |

What is claimed is:

1. An apparatus for posture recognition comprising:
a triangulation unit for dividing a posture of a body into a plurality of triangular meshes; and
a recognition unit for forming a spanning tree correspond to the meshes to recognize the posture.

2. The apparatus for posture recognition according to claim 1 further comprises a background subtraction unit to extract and define a boundary of the body.

3. The apparatus for posture recognition according to claim 2, wherein the background subtraction unit is a video.

4. The apparatus for posture recognition according to claim 1, wherein the meshes are triangles.

5. The apparatus for posture recognition according to claim 1, wherein the recognition unit is achieved via a skeleton analysis or a centroid context analysis.

6. The apparatus for posture recognition according to claim 5, wherein the skeleton analysis is defined via a graph search scheme.

7. An apparatus for behavior analysis comprising:
a clustering unit for key postures selection via merging a plurality of postures iteratively;
a coding unit for translating all the input postures into a plurality of correspondent symbols according to the selected key postures; and
a matching unit, which takes advantages of the coding unit, for unscrambling the correspondent symbols to distinguish a behavior.

8. The apparatus for behavior analysis according to claim 7, wherein the clustering unit is programmable.

9. The apparatus for behavior analysis according to claim 7, wherein the clustering unit is user-defined.

10. The apparatus for behavior analysis according to claim 7, wherein the postures are obtained from an apparatus for posture recognition comprising:
a triangulation unit for dividing a body posture into a plurality of triangular meshes; and
a recognition unit for forming a spanning tree from the meshes to recognize the posture.

11. The apparatus for behavior analysis according to claim 10, wherein the apparatus for posture recognition further comprises a background subtraction unit to extract and define boundaries of the body.

12. The apparatus for behavior analysis according to claim 10, wherein the meshes can be defined via a triangle-mesh algorithm.

13. The apparatus for behavior analysis according to claim 10, wherein the recognition unit is achieved via a skeleton analysis or a centroid context analysis.

14. The apparatus for behavior analysis according to claim 13, wherein the skeleton analysis is defined via a graph search scheme.

15. The apparatus for behavior analysis according to claim 13, wherein the centroid context is formed by labeling each mesh with a number.

16. The apparatus for behavior analysis according to claim 7, wherein the correspondent symbols are unscrambling via a string matching method.

17. A method for posture recognition, comprising the steps of:
triangulating a posture of a body into a plurality of triangular meshes;
forming a skeleton analysis and a centroid context analysis correspond to the triangulated meshes; and
recognizing and analyzing the posture.

18. The method for posture recognition according to claim 17, wherein extracting and defining a boundary of the body is done via a background subtraction.

19. The method for posture recognition according to claim 17, wherein forming the meshes is based on a general triangulation algorithm.

20. The method for posture recognition according to claim 17, wherein the skeleton analysis comprises the steps of:
inputting a set of the triangle meshes extracted from the posture;
constructing a graph from the set of triangle meshes according to connectivity of a plurality of nodes in the triangle meshes;
applying a depth first search to the graph for finding a correspondent spanning tree;
extracting the skeleton feature from the spanning tree; and
outputting the skeleton feature of the posture.

21. The method for posture recognition according to claim 17, wherein a distance between the two different postures, P and D, defined via the skeleton analysis is satisfied with:
$${d}_{skeleton} ( {S}_{P} , {S}_{D} ) = \frac{1}{\vert {DT}_{{S}_{P}} \vert} {\sum }_{r} \vert {DT}_{{S}_{P}} ( r ) - {DT}_{{S}_{D}} ( r ) \vert ,$$
where S_{P}, and S_{D} are the skeletons correspond to the postures P and D.

22. The method for posture recognition according to claim 17, wherein the centroid context analysis comprises the steps of:
finding the spanning tree of the posture;
tracing the spanning tree via using the depth first search recursively until the spanning tree is empty;
removing a plurality of branch nodes from the spanning tree;
finding and collecting a plurality of visited nodes from a set of paths;
defining a centroid histogram of each path centroid via using:
hᵣ(k)=#{q|q≠r, (q−r)εbin^{k}}, where bin^{k} is a kth bin of log-polar coordinate; and
collecting all the histograms as the output of the centroid context extraction of the posture.

23. The method for posture recognition according to claim 17, wherein a distance between the two different postures, P and Q, defined via the centroid context analysis is satisfied with:
$${d}_{cc} ( P , Q ) = \frac{1}{2 \vert {V}^{P} \vert} {\sum }_{i = 0}^{\vert {V}^{P} \vert - 1} {w}_{i}^{P} {min}_{0 \leq j < \vert {V}^{P} \vert} C ( {c}_{i}^{P} , {c}_{j}^{Q} ) + \frac{1}{2 \vert {V}^{Q} \vert} {\sum }_{j = 0}^{\vert {V}^{Q} \vert - 1} {w}_{j}^{Q} {min}_{0 \leq i < \vert {V}^{Q} \vert} C ( {c}_{i}^{P} , {c}_{j}^{Q} ) ,$$
where V^{P} and V^{Q} are the path centroids for the posture P and Q while wᵢ^{P} and wⱼ^{Q} are area ratios of the ith and jth parts of the posture P and Q.

24. The method for posture recognition according to claim 17, wherein a distance between the two different postures, P and Q, defined via both the skeleton analysis and the centroid context analysis is satisfied with:
Error(P,Q)=w dₛₖₑₗₑₜₒₙ(P,Q)+(1−w)d_{CC}(P,Q),
where Error(P,Q) is the integrated distance between the postures P and Q and w is a weight for balancing the two distances dₛₖₑₗₑₜₒₙ(P,Q) and d_{CC}(P,Q).

25. A method for behavior analysis, comprising the steps of:
selecting a plurality of key postures;
coding the input postures into a plurality of correspondent symbols according to the selected key postures; and
matching the correspondent symbols to distinguish a behavior.

26. The method for behavior analysis according to claim 25, wherein selecting the key postures is programmable.

27. The method for behavior analysis according to claim 25, wherein selecting the key postures is user-defined.

28. The method for behavior analysis according to claim 25, wherein selecting the key postures is via clustering a plurality of time-varied postures.

29. The method for behavior analysis according to claim 28, wherein a distance between the two selected cluster elements, zᵢ and zⱼ, is satisfied with:
$${d}_{cluster} ( {z}_{i} , {z}_{j} ) = \frac{1}{\vert {z}_{i} \vert \vert {z}_{j} \vert} {\sum }_{{e}_{m} ∈ {z}_{i}} {\sum }_{{e}_{n} ∈ {z}_{j}} Error ( {e}_{m} , {e}_{n} ) ,$$
where |zₖ| is absolute value of the cluster elements in zₖ.

30. The method for behavior analysis according to claim 28, wherein the key posture is satisfied with:
$${e}_{i}^{key} = arg {min}_{{e}_{m} ∈ {\overline{z}}_{i}} {\sum }_{{e}_{n} ∈ {\overline{z}}_{i}} Error ( {e}_{m} , {e}_{n} ) .$$

31. The method for behavior analysis according to claim 28, wherein the matching step is based on a string matching method.

32. The method for behavior analysis according to claim 31, wherein the string matching method comprises inserting, deleting and replacing.

33. The method for behavior analysis according to claim 31, wherein an edit distance between S_{Q}[0 . . . i] and S_{D}[0 . . . j] of two strings S_{Q} and S_{D} based on the string matching method is defined as:
D_{S}_{_{Q}}_{,S}_{_{D}}^{e}(i,j)=min[D_{S}_{_{Q}}_{,S}_{_{D}}^{e}(i−1,j)+C_{i,j}^{I}), D_{S}_{_{Q}}_{,S}_{_{D}}^{e}(i,j−1)+C_{i,j}^{D}), D_{S}_{_{Q}}_{,S}_{_{D}}^{e}(i−1,j−1)+α(i−1,j−1)],
where C_{i,j}^{I}=ρ+(1−ρ)α(i−1,j), C_{i,j}^{D}=ρ+(1−ρ)α(i,j−1) and ρ is smaller than 1.

34. A system for irregular human action analysis comprising:
an action recognition apparatus for integrating a plurality of postures to define an action; and
a judging apparatus for identifying whether the action is irregular.

35. The system for irregular human action analysis according to claim 34, wherein the analyzing apparatus comprises:
a posture recognition apparatus for recognizing the individual posture; and
a behavior recognition apparatus for distinguishing a behavior via a plurality of key postures selected from the postures.

36. The system for irregular human action analysis according to claim 35, wherein the posture recognition apparatus comprises:
a triangulation unit for dividing the posture of a body into a plurality of triangular meshes; and
a recognition unit for forming a spanning tree correspond to the meshes to recognize the posture.

37. The system for irregular human action analysis according to claim 35, the posture recognition apparatus further comprises a background subtraction unit to extract and define a boundary of the body.

38. The system for irregular human action analysis according to claim 37, wherein the background subtraction unit is a video.

39. The system for irregular human action analysis according to claim 35, wherein the meshes are triangles.

40. The system for irregular human action analysis according to claim 35, wherein the recognition unit is achieved via a skeleton analysis or a centroid context analysis.

41. The system for irregular human action analysis according to claim 35, wherein the skeleton analysis is defined via a graph search scheme.

42. The system for irregular human action analysis according to claim 34, wherein the behavior recognition apparatus comprises:
a clustering unit for selecting the key postures via clustering the postures iteratively for defining the various regular behaviors/actions;
a coding unit for translating the input postures into a plurality of correspondent symbols according to the selected key postures; and
a matching unit for unscrambling the correspondent symbols to distinguish the irregular/suspicious behavior.

43. The system for irregular human action analysis according to claim 42, wherein the clustering unit is programmable.

44. The system for irregular human action analysis according to claim 42, wherein the clustering unit is user-defined.

45. The system for irregular human action analysis according to claim 42, wherein the correspondent symbols are unscrambling via a string matching method.

46. The system for irregular human action analysis according to claim 42, wherein the matching unit is achieved via a symbol counting method for finding a series of irregular/suspicious posture patterns from input video sequences using the set of key postures.

47. The system for irregular human action analysis according to claim 34 further comprises a warning unit for sending an alarm if the behavior is irregular.

48. The system for irregular human action analysis according to claim 47, wherein the alarm is sent to a surveillance system.

49. The system for irregular human action analysis according to claim 47, wherein the warning unit is selected from an audio media, a color-highlighted video media or a light-emitted media.

50. A method for irregular human action analysis, comprising the steps of:
calculating the distance between a posture P and a set K of a plurality of selected key postures with:
$$d ( P , K ) = {max}_{Q ∈ K} d is ( P , q ) ;$$
and
judging the posture P as a irregular posture if d(P,Q) is larger than a threshold.

51. The method for irregular human action analysis according to claim 50, wherein the threshold is programmable.

52. The method for irregular human action analysis according to claim 50, wherein the threshold is user-defined.

53. The method for irregular human action analysis according to claim 50, wherein defining the distance, dis(P,Q), between the two different postures, P and Q, is selected from the methods of a skeleton analysis or a centroid context analysis.

54. The method for irregular human action analysis according to claim 50, wherein the distance, dis(P,Q), between the two different postures, P and Q, defined via the skeleton analysis is satisfied with:
$${d}_{skeleton} ( {S}_{P} , {S}_{Q} ) = \frac{1}{\vert {DT}_{{S}_{P}} \vert} {\sum }_{r} \vert {DT}_{{S}_{P}} ( r ) - {DT}_{{S}_{Q}} ( r ) \vert ,$$
where S_{P} and S_{Q} are the skeletons correspond to the postures P and Q.

55. The method for irregular human action analysis according to claim 50, wherein the distance dis(P,Q), between the two different postures, P and Q, defined via the centroid context analysis is satisfied with:
$${d}_{cc} ( P , Q ) = \frac{1}{2 \vert {V}^{P} \vert} {\sum }_{i = 0}^{\vert {V}^{P} \vert - 1} {w}_{i}^{P} {min}_{0 \leq j < \vert {V}^{P} \vert} C ( {c}_{i}^{P} , {c}_{j}^{Q} ) + \frac{1}{2 \vert {V}^{Q} \vert} {\sum }_{j = 0}^{\vert {V}^{Q} \vert - 1} {w}_{j}^{Q} {min}_{0 \leq i < \vert {V}^{Q} \vert} C ( {c}_{i}^{P} , {c}_{j}^{Q} ) ,$$
where V^{P} and V^{Q} are the path centroids for the posture P and Q while wᵢ^{P} and wⱼ^{Q} are area ratios of the ith and jth parts of the posture P and Q.

56. The method for irregular human action analysis according to claim 50, wherein the distance dis(P,Q), between the two different postures, P and Q, defined via both the skeleton analysis and the centroid context analysis is satisfied with:
Error(P,Q)=wdₛₖₑₗₑₜₒₙ(P,Q)+(1−w)d_{CC}(P,Q),
where Error(P,Q) is the integrated distance between the postures P and Q and w is a weight for balancing the two distances dₛₖₑₗₑₜₒₙ(P,Q) and d_{CC}(P,Q).
